(19) United States
(12) Patent Application Publication
(10) Pub. No.: US 2022/0023465 A1
(43) Pub. Date: Jan. 27, 2022

(54) SYSTEM FOR DISINFECTING A POINT-OF-SALE TERMINAL

(71) Applicant: Justin HARTENSTEIN, Metairie, LA (US)

(72) Inventor: Justin HARTENSTEIN, Metairie, LA (US)

(21) Appl. No.: 17/376,477

(22) Filed: Jul. 15, 2021

Related U.S. Application Data
(60) Provisional application No. 63/056,672, filed on Jul. 26, 2020.

(51) Int. Cl. A61L 2/10; G07F 19/00; A61L 2/26; G07F 7/08; G07F 7/00

(52) U.S. Cl. CPC A61L 2/10; G07F 19/201; G07F 19/205; A61L 2202/14; G07F 7/088; G07F 7/005; A61L 2202/20; A61L 2/26

(57) ABSTRACT

It is described herein a system for disinfecting a point-of-sale terminal. The system may comprise a housing and at least one light source. The housing may be configured to connect to a first exterior surface of the point-of-sale terminal with the at least one light source mounted within a hollow interior of the housing. The at least one light source may be configured to project light in the ultraviolet spectrum of between 100 nm and 400 nm at a first angle relative to a first plane of the point-of-sale terminal upon receiving an electrical signal from a switching mechanism. The first angle may be in the range of between 0° and 90°.

### CROSS REFERENCES AND PRIORITIES

This application claims priority from U.S. Provisional Application No. 63/056,672 filed on 26 Jul. 2021 the teachings of which are incorporated by reference herein in their entirety.

### BACKGROUND

Point-of-sale terminals—such as credit card readers and Automatic Teller Machines (ATMs)—exist in a variety of different locations. Nonlimiting examples include retail check out counters, gas station pumps, and vending machines. While point-of-sale terminals provide an easy mechanism of payment for goods and services, their use by multiple persons over a period of time can result in contamination with a wide variety of bacterial and viral infectious agents.

Disinfecting these high touch surfaces on point-of-sale terminals often involves additional human interaction. In the typical scenario, a person periodically sprays and wipes the surfaces of the point-of-sale terminal with a liquid containing an active agent such as hydrogen peroxide, benzalkonium chloride, or sodium hypochlorite which is known to kill or deactivate bacteria and viruses. This process may be repeated at different intervals ranging from after every purchase to at the end of every business day or even weekly or monthly.

The process of spraying and wiping the surfaces of a point-of-sale terminal is inefficient and often ineffective. When the person assigned to conduct these operations fails to do so at the assigned intervals, infectious agents can build up on the high touch surfaces of the point-of-sale terminal resulting in increased risk of transmitting infections to later users. Additionally, the process of spraying and wiping can be ineffective if the person assigned to conduct these operations does not diligently spray and wipe all surfaces of the point-of-sale terminal. Even when the person spraying and wiping the point-of-sale terminal is diligent in performing their job duties, the process requires time which can cause delays in processing future transactions.

Attempts have been made to improve the process of disinfecting point-of-sale terminals by reducing or removing the need for human interaction in the disinfecting process. For instance, attempts have been made to disinfect a wide variety of surfaces using ultraviolet light. Ultraviolet light is known to kill or inactivate microorganisms by destroying nucleic acids which, in turn, disrupts their DNA, rendering them inactive by leaving these cells unable to perform vital cellular functions. While ultraviolet light disinfects for microorganisms such as bacteria and viruses, its effects can also damage human cells and degrade surface materials such as plastics, woods, and even metals.

One attempt to disinfect surfaces using ultraviolet light is disclosed in United States Patent Publication No. 2018/0339075 A1 which discloses that a, “ . . . surface treatment unit can include a plurality of disinfection illuminators with each configured to irradiate one of the user interactive components of the ATM with ultraviolet radiation.” (internal citations omitted). US 2018/0339075 A1 indicates that the disinfection illuminators include sensors, “ . . . used to detect the conditions (e.g., bacterial conditions) present at the surfaces of the various user-interactive components and send signals representative of the conditions to the control units. Each respective control unit can monitor the conditions and determine if the signals are indicative of conditions that are sufficient to warrant a disinfection treatment.” (internal citations omitted).

In practice, the systems disclosed in US 2018/0339075 A1 are often ineffective as they rely upon the sensor and control unit to detect the presence of a sufficient amount of bacteria in order to activate the ultraviolet disinfecting system. These sensors and control units can often fail, resulting in the ultraviolet disinfecting lights not being activated. Even when the sensors and control unit work correctly, they only activate the ultraviolet disinfecting lights once a threshold level of bacteria is detected. This may be ineffective in protecting against buildup of viruses on the point-of-sale terminal. In addition, only activating the ultraviolet disinfecting lights after a threshold level of bacteria is detected also leaves users of the point-of-sale terminal exposed to a certain level of bacteria and viruses on the point-of-sale terminal surfaces between disinfecting cycles. Additionally, by focusing only on the interactive components, the system disclosed in US 2018/0339075 A1 may fail to disinfect other surfaces of the point-of-sale terminal which may not be high touch surfaces, but may nevertheless come into contact with humans using the point-of-sale terminal.

The need exists, therefore, for an improved system and method for disinfecting surfaces of a point-of-sale terminal using ultraviolet light.

### SUMMARY

Disclosed herein is a system for disinfecting a point-of-sale terminal. The point-of-sale terminal may comprise at least one planar display screen and optionally at least one keypad.

The system may comprise a housing and at least one light source. The housing may comprise a first end wall and a second end wall. The housing may also comprise a first longitudinal wall spanning a distance between a first end wall first edge and a second end wall first edge. The housing may further comprise a second longitudinal wall spanning a distance between a first end wall second edge and a second end wall second edge. The housing may also comprise an opening extending from a second longitudinal wall first edge. The housing may be configured to connect to a first exterior surface of the point-of-sale.

The at least one light source may be mounted within a hollow interior of the housing. The at least one light source may also be configured within the hollow interior of the housing to project light at a first angle relative to a first plane of the at least one planar display screen and/or the at least one keypad when the housing is connected to the first exterior surface of the point-of-sale terminal. The first angle may be in a range of between 0° and 90°.

The at least one light source may be electrically connected to a switching mechanism. The at least one light source may be capable of emitting light in the ultraviolet spectrum of between 100 nm and 400 nm upon receiving an electrical signal from the switching mechanism.

In some embodiments, the at least one light source may comprise at least one light-emitting diode. In certain embodiments, the at least one light source may comprise a plurality of light sources. In some embodiments, the at least one light source may be capable of emitting light in the ultraviolet spectrum of between 200 nm and 300 nm upon receiving the electrical signal from the switching mechanism.

In certain embodiments, the system may further comprise a stylus holder. The stylus holder may be configured to connect to a second exterior surface of the point-of-sale terminal. The stylus holder may comprise a first end, and a second end opposite the first end. The stylus holder may also comprise a sidewall spanning a distance between the first end and the second end. The stylus holder may further comprise a first end access point, and a cavity originating from the first end access point. The cavity may be configured to receive a stylus. The stylus holder may also comprise at least one secondary light source.

In embodiments comprising a stylus holder, the at least one secondary light source may be mounted within the cavity. The at least one secondary light source may also be electrically connected to the switching mechanism. The at least one secondary light source may be capable of emitting light in the ultraviolet spectrum of between 100 nm and 400 nm upon receiving an electrical signal from the switching mechanism.

In some embodiments comprising a stylus holder, the system may further comprise a secondary switching mechanism. The at least one secondary light source may be electrically connected to the secondary switching mechanism. The at least one secondary light source may be capable of emitting light in the ultraviolet spectrum of between 100 nm and 400 nm upon receiving an electrical signal from the secondary switching mechanism.

In certain embodiments comprising a stylus holder, the secondary switching mechanism may be contained within the point-of-sale terminal. The secondary switching mechanism may be configured to send an electrical signal turning on the at least one secondary light source upon completion of a transaction conducted on the point-of-sale terminal.

In some embodiments comprising a stylus holder, the secondary switching mechanism may comprise a sensor. The sensor may be configured to send an electrical signal turning on the at least one secondary light source when the sensor detects the presence of the stylus within the cavity.

In certain embodiments comprising a stylus holder, the secondary switching mechanism may comprise a pressure switch. The pressure switch may be located within the cavity. The pressure switch may be configured to send an electrical signal turning on the at least one secondary light source when a pressure is applied to the pressure switch by the stylus.

In some embodiments comprising a stylus holder, the secondary switching mechanism may comprise a secondary passive infrared sensor. The secondary passive infrared sensor may be configured to send an electrical signal turning off the at least one secondary light source when the secondary passive infrared sensor detects the presence of a person in the vicinity of the point-of-sale terminal.

In certain embodiments comprising a stylus holder, the secondary switching mechanism may comprise a timer. The timer may be configured to turn off the at least one secondary light source after a first period of time. The first period of time may be in the range of between 10 seconds after the at least one secondary light source is turned on and 30 minutes after the at least one secondary light source is turned on.

In some embodiments comprising a stylus holder, the at least one secondary light source may comprise at least one secondary light-emitting diode. In certain embodiments, the at least one secondary light source may be capable of emitting light in the ultraviolet spectrum of between 200 nm and 300 nm upon receiving the electrical signal from the switching mechanism.

Certain embodiments of the point-of-sale terminal may further comprise a stylus clip. In some such embodiments, the stylus holder may be configured to be held within the stylus clip.

In some embodiments comprising a stylus holder, the sidewall may comprise an adhesive exterior surface. In other embodiments, the stylus holder may be molded to the second exterior surface.

In certain embodiments, the switching mechanism may be contained within the point-of-sale terminal. The switching mechanism may be configured to send an electrical signal turning on the at least one light source upon completion of a transaction conducted on the point-of-sale terminal.

In some embodiments, the switching mechanism may comprise a passive infrared sensor. The passive infrared sensor may be configured to send an electrical signal turning off the at least one light source when the passive infrared sensor detects the presence of a person in the vicinity of the point-of-sale terminal.

In certain embodiments, the switching mechanism may comprise a timer. The timer may be configured to turn off the at least one light source after a first period of time. The first period of time may be in the range of between 10 seconds after the at least one light source is turned on and 30 minutes after the at least one light source is turned on.

In some embodiments, the housing may comprise a bottom wall. The bottom wall may comprise an adhesive exterior surface. In certain embodiments, the housing may comprise an extension wall comprising an adhesive surface. In other embodiments, the housing may be molded to the first exterior surface.

### BRIEF DESCRIPTION OF FIGURES

FIG. 1 is an exploded perspective view one embodiment of a system for disinfecting a point-of-sale terminal.

FIG. 2 is a perspective view of a housing for an embodiment of a system for disinfecting a point-of-sale terminal.

FIG. 3 is a perspective view of a stylus holder for an embodiment of a system for disinfecting a point-of-sale terminal.

FIG. 4 is an assembled perspective view of the embodiment of a system for disinfecting a point-of-sale terminal shown in FIG. 1.

FIG. 5 is a cross-section side view of an embodiment of a system for disinfecting a point-of-sale terminal.

FIG. 6 is a cross-section view of an embodiment of a stylus holder for a system for disinfecting a point-of-sale terminal.

### DETAILED DESCRIPTION

Disclosed herein is a system for disinfecting a point-of-sale terminal. The system is described below with reference to the Figures. As described herein and in the claims, the following numbers refer to the following structures as noted in the Figures.
10 refers to a point-of-sale terminal.
12 refers to a planar display screen (of the point-of-sale terminal).
16 refers to a first exterior surface (of the point-of-sale terminal).
18 refers to a second exterior surface (of the point-of-sale terminal).
100 refers to a housing.
110 refers to a first end wall (of the housing).
111 refers to a first end wall first edge.
112 refers to a first end wall second edge.
120 refers to a second end wall (of the housing).
121 refers to a second end wall first edge.
122 refers to a second end wall second edge.
130 refers to a first longitudinal wall (of the housing).
140 refers to a second longitudinal wall (of the housing).
141 refers to second longitudinal wall first edge.
150 refers to an opening (in the housing).
160 refers to a bottom wall (of the housing).
200 refers to a light source.
210 refers to a secondary light source.
300 refers to a stylus holder.
310 refers to a first end (of the stylus holder).
315 refers to a first end access point.
320 refers to a second end (of the stylus holder).
330 refers to a sidewall (of the stylus holder).
340 refers to a cavity.
400 refers to a stylus.
ω₁ refers to a first angle.

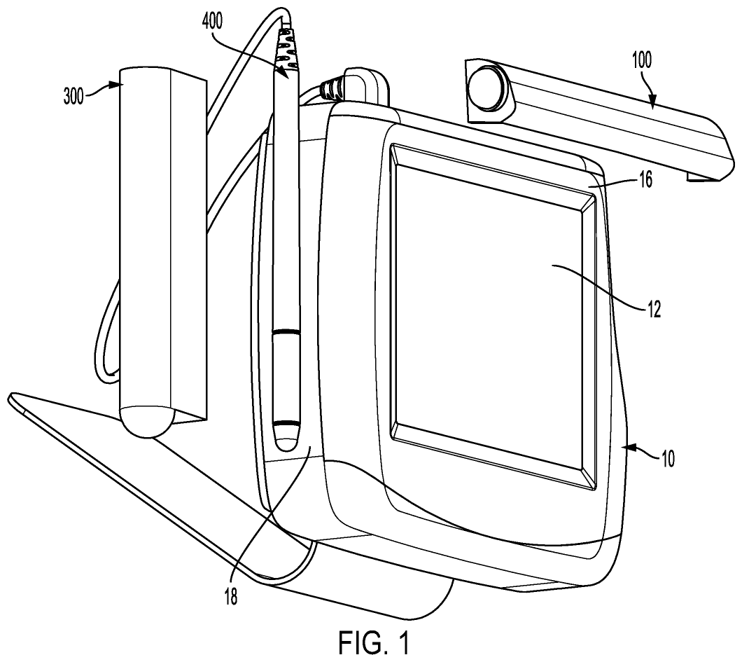

FIG. 1 depicts an exploded perspective view of one embodiment of a system for disinfecting a point-of-sale terminal (10). A point-of-sale terminal may be any electronic equipment having interactive components used to facilitate aspects of a transaction. Specific nonlimiting examples of point-of-sale terminals include automatic teller machines (ATM), credit card readers, and user-interactive screens at retail displays. Credit card readers in particular may exist in a variety of locations. Specific nonlimiting examples of such locations include retail checkout counters, gas station pumps, parking meters, and toll booths. As used herein and in the claims, the term “point-of-sale terminal” is intended to encompass all such point-of-sale terminals and any variations of such point-of-sale terminals.

As shown in FIG. 1, the point-of-sale (10) terminal may comprise at least one planar display screen (12). In certain embodiments, the point-of-sale terminal may comprise a plurality of planar display screens. A planar display screen may be an electronic component of the point-of-sale terminal which shows details of the transaction such as an itemized list of the goods or services being purchased and/or the monetary amount of the purchase. In some embodiments, the planar display screen may be an interactive planar display screen which allows the user to enter information such as a personal identification number (PIN), signature, or the monetary value of a gratuity into the point-of-sale terminal using their finger or a stylus.

Optionally, the point-of-sale terminal (10) may also comprise at least one keypad (not shown). A keypad may comprise a plurality of capacitive touch keys arranged on a circuit board within the point-of-sale terminal. Often, at least some of the capacitive touch keys will correspond to the numbers 0 through 9. These capacitive touch keys allow the user to enter information such as a personal identification number (PIN) or the monetary value of a gratuity into the point-of-sale terminal using their finger to press the capacitive touch keys.

The point-of-sale terminal (10) may comprise a plurality of exterior surfaces. At least a portion of the exterior surfaces of the point-of-sale terminal may be used as a surface to which a component of the system for disinfecting a point-of-sale terminal is connected. For example, a first exterior surface (16) of the point-of-sale terminal may be a surface to which a housing (100) is connected. In some embodiments, a second exterior surface (18) of the point-of-sale terminal may be a surface to which a stylus holder (300) is connected.

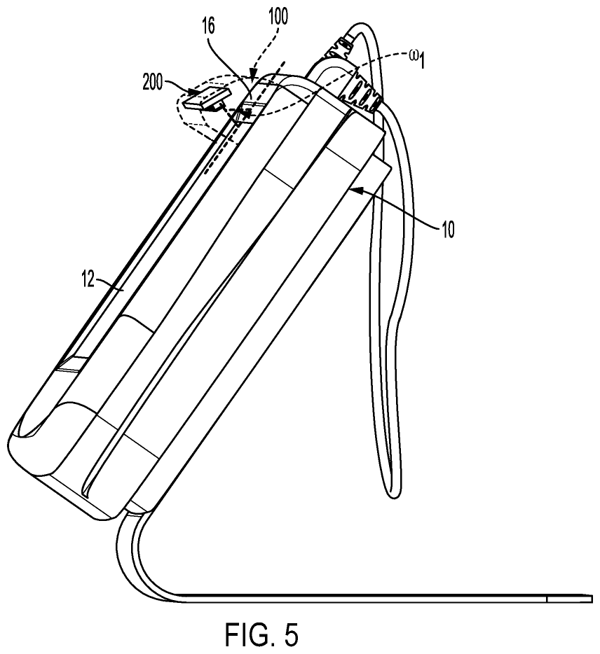

The system for disinfecting a point-of-sale terminal (10) may comprise a housing (100) as shown in FIG. 1 and at least one light source (200 as shown in FIG. 5). The at least one light source may be mounted within a hollow interior of the housing. The at least one light source may be configured within the hollow interior of the housing to project light at a first angle ((ω₁) as shown in FIG. 5) relative to a first plane of the at least one planar display screen (12) and/or the optional at least one keypad when the housing is connected to the first exterior surface (16) of the point-of-sale terminal.

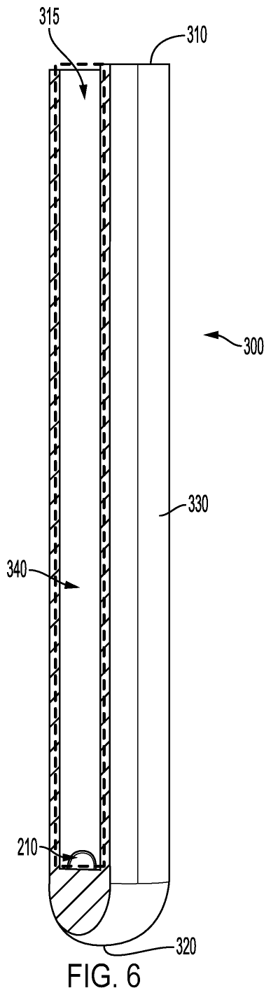

In some embodiments, the system for disinfecting a point-of-sale terminal (10) may also comprise a stylus holder (300) as shown in FIG. 1. A stylus holder may be a device capable of housing a stylus (400) while the stylus is not in use by a user. In general, the stylus holder will comprise a cavity ((340) as shown in FIG. 6) surrounded by at least one sidewall ((330) as shown in FIG. 6).

Certain point-of-sale terminals may already include an existing stylus clip. A stylus clip generally consists of two opposing pieces of deformable material such as plastic with the distance between the two pieces of deformable material being slightly smaller than the diameter of the stylus. As the stylus is pressed into the gap between the two pieces of deformable material, the materials deflect in opposing directions. Once the stylus is fully disposed into the gap, the two pieces of material retract to provide opposing forces against the stylus which hold the stylus in place. In some embodiments, the stylus holder (300) may be configured to fit within the gap of the existing stylus clip such that the stylus holder is connected to the point-of-sale terminal (10) by the existing stylus clip.

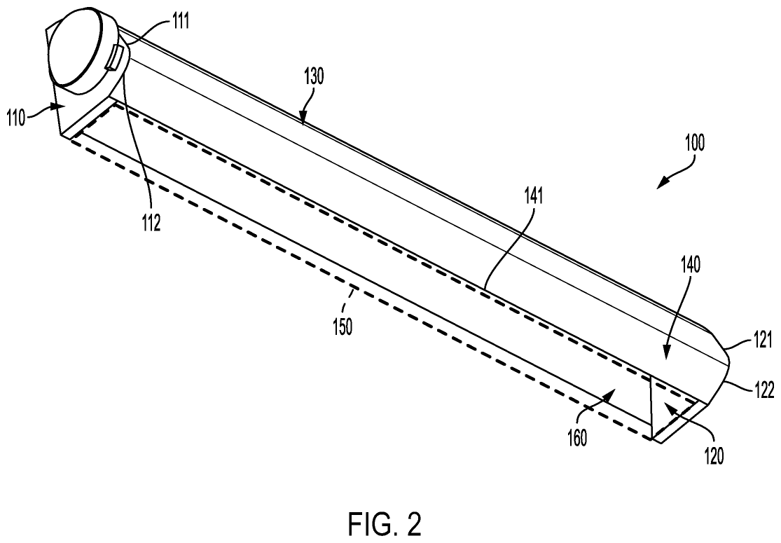

FIG. 2 shows a perspective view of a housing (100) for a system for disinfecting a point-of-sale terminal. As shown in FIG. 2, the housing may comprise a first end wall (110) and a second end wall (120) which is opposite the first end wall. The housing may also comprise at least one longitudinal wall spanning the distance between and connecting to corresponding edges of the first end wall and the second end wall.

The at least one longitudinal wall may comprise a plurality of longitudinal walls consisting of at least a first longitudinal wall (130) and a second longitudinal wall (140). The first longitudinal wall may span a distance between a first end wall first edge (111) and a second end wall first edge (121) as shown in FIG. 2. The second longitudinal wall may span a distance between a first end wall second edge (112) and a second end wall second edge (122) as shown in FIG. 2.

In some embodiments, the at least one longitudinal wall may be a single wall having a curved profile and extending between a curved edge of the first end wall and a curved edge of the second end wall.

FIG. 2 also shows the housing having an optional bottom wall (160). The bottom wall as shown in FIG. 2 is a longitudinal wall extending between the first end wall and the second end wall. In the embodiment shown, the bottom wall may comprise an adhesive exterior surface. The adhesive exterior surface—when present—may be used to connect the housing to the first exterior surface ((16 as shown in FIG. 1) of the point-of-sale terminal ((10) as shown in FIG. 1).

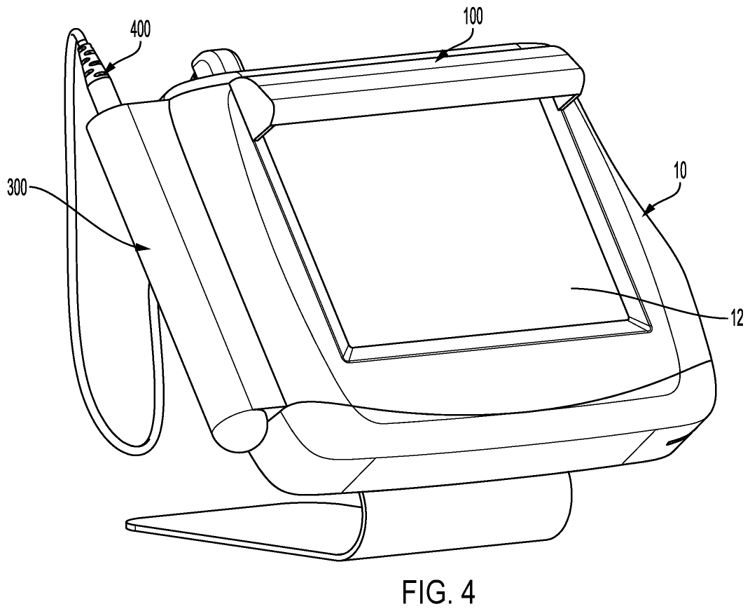

The shield may also comprise an opening (150) as shown in FIG. 2. The opening will have a perimeter defined by at least one edge of the first end wall (110), the second end wall (120), and at least one of the longitudinal wall(s). For example, the opening may extend from a second longitudinal wall first edge (141) as shown in FIG. 2. The opening allows light emitted from the at least one light source (200 as shown in FIG. 5) to be projected out of the housing in the direction of the point-of-sale terminal ((10 as shown in FIG. 4) as described herein.

In certain embodiments, the housing (100) may also comprise an extension wall. An extension wall—when present—is a planar surface attached to and extending away from the housing. When used, the extension wall may comprise an adhesive surface. This adhesive surface—when present—may be used to connect the housing to the first exterior surface of the point-of-sale terminal. In such embodiments, the first exterior surface of the point-of-sale terminal may be an edge surface of the point-of-sale terminal.

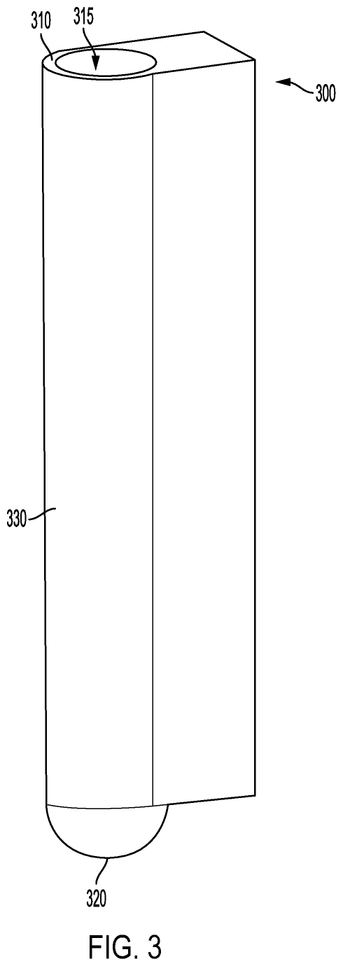

FIG. 3 depicts a perspective view of a stylus holder (300). As shown in FIG. 3, the stylus holder may comprise a first end (310), a second end (320) which is opposite of the first end, and a sidewall (330) which may span the distance between the first end and the second end. As also shown in FIG. 3, the stylus holder may comprise a first end access point (315) to a cavity (340 as shown in FIG. 6). As described herein, the cavity may hold the stylus ((400) as shown in FIG. 1) while it is not in use by passing the stylus into the cavity through the first end access point.

FIG. 4 depicts a perspective view of the system for disinfecting a point-of-sale terminal of FIG. 1 in assembled form. FIG. 4 shows the point-of-sale terminal (10) with its planar display screen (12). Also shown in FIG. 4 is the housing (100) which is connected to the first exterior surface (16 as shown in FIG. 1). FIG. 4 further shows the optional stylus holder (300) which is connected to the second exterior surface (18 as shown in FIG. 1).

As described herein, one method of connecting the housing (100) to the first exterior surface (16) of the point-of-sale terminal (10) may be by way of an adhesive. Other embodiments may include the use of fasteners such as screws or rivets used in addition to or instead of an adhesive. Adhesives and fasteners may be used in retrofit applications in which the system for disinfecting a point-of-sale terminal is installed onto an existing point-of-sale terminal. However, adhesives and fasteners may also be used in applications with new point-of-sale terminals. An alternative embodiment for connecting the housing to the first exterior surface in new point-of-sale terminals may involve manufacturing the housing and at least one exterior component of the point-of-sale terminal from a single unitary piece of material such that the housing is integrally connected to the first exterior surface.

As described herein, one method of connecting the stylus holder (300) to the second exterior surface (18) of the point-of-sale terminal (10) may be by way of an adhesive. Other embodiments may include the use of fasteners such as screws or rivets used in addition to or instead of an adhesive. Adhesives and fasteners may be used in retrofit applications in which the system for disinfecting a point-of-sale terminal is installed onto an existing point-of-sale terminal. However, adhesives and fasteners may also be used in applications with new point-of-sale terminals. An alternative embodiment for connecting the stylus holder to the second exterior surface in new point-of-sale terminals may involve manufacturing the stylus holder and at least one exterior component of the point-of-sale terminal from a single unitary piece of material such that the stylus holder is integrally connected to the second exterior surface. Another method for connecting the stylus holder to the point-of-sale terminal may involve holding the stylus holder within a stylus clip as described herein.

Depicted in FIG. 5 is a cross section side view of an embodiment of a system for disinfecting a point-of-sale terminal. FIG. 5 shows the point-of-sale terminal (10) with its planar display screen (12). FIG. 5 also shows the housing (100) connected to the first exterior surface (16) of the point-of-sale terminal with a light source (200) mounted within the hollow interior of the housing.

As depicted in FIG. 5, the light source is configured within the hollow interior of the housing to project light at a first angle (ω₁) relative to a first plane of the at least one planar display screen and/or the optional at least one keypad when the housing is connected to the first exterior surface (16) of the point-of-sale terminal (10). The first angle may be in a range selected from the group consisting of between 0° and 90°, between 0° and 75°, between 0° and 60°, between 0° and 45°, between 0° and 30°, between 0° and 15°, between 15° and 90°, between 15° and 75°, between 15° and 60°, between 15° and 45°, between 15° and 30°, between 30° and 90°, between 30° and 75°, between 30° and 60°, between 30° and 45°, between 45° and 90°, between 45° and 75°, between 45° and 60°, between 60° and 90°, between 60° and 75°, and between 75° and 90°.

The first angle may be varied to adjust for point-of-sale terminals having different exterior profiles. The exterior profile of the point-of-sale terminal refers—generally—to the shape and size of the point-of-sale terminal, but also refers to the location, shape and size of different features of the point-of-sale terminal including the planar display screen and/or the size and configuration of various components of the keypad. For example, point-of-sale terminals having a larger exterior profile—such as those associated with an ATM—may utilize a larger first angle, such as a first angle in the range of between 60° and 75° to provide greater exposure of the point-of-sale terminals exterior surfaces to light emitted from the at least one light source. Point-of-sale terminals having a smaller exterior profile—such as those associated with a retail store credit card reader—may utilize a smaller first angle, such as a first angle in the range of between 15° and 30° to concentrate the exposure of light emitted from the at least one light source onto the smaller exterior profile of such point-of-sale terminals. In some embodiments, the first angle may be adjustable within the system by connecting one or both of the housing and/or the at least one light source to the first exterior surface of the point-of-sale terminal in a pivotable manner.

FIG. 6 shows a cross section side view of an embodiment of a stylus holder (300) specifically illustrating the cavity (340). As shown in FIG. 6, at least one secondary light source (210) may be mounted within the cavity. The secondary light source may be mounted at any location within the cavity including proximate to the first end (310), proximate to the second end (320), or at any point along an inner sidewall of the cavity.

The at least one light source mounted within the housing may be any type of light source capable of emitting light in the ultraviolet spectrum. Preferably, the light source is designed to create a wavelength of light having disinfecting characteristics. This type of disinfecting light is commonly known as ultraviolet germicidal irradiation (UVGI). UVGI is a disinfection method that uses short-wavelength ultraviolet A (UVA), ultraviolet B (UVB), and/or ultraviolet C (UVC) light to kill or inactivate microorganisms by destroying nucleic acids which, in turn, disrupts their DNA, rendering them inactive by leaving these cells unable to perform vital cellular functions.

The UVGI light produced by the light source may aid in sterilization of the surfaces of the point-of-sale terminal. The irradiation of the UVGI light utilizes photons to disinfect the surfaces. UVGI light damages a pathogen's DNA or RNA, which prevents it from replicating and infecting the body.

UV light is electromagnetic radiation with wavelengths shorter than visible light, but longer than X-rays. UV can be separated into various ranges, with short-wavelength UVC generally considered to be “germicidal UV”. Wavelengths between about 200 nm and 300 nm are strongly absorbed by nucleic acids. The absorbed energy can result in defects including pyrimidine dimers. These dimers can prevent replication or prevent the expression of necessary proteins, resulting in the death or inactivation of the organism. However, light sources emitting UV light in a range selected from the group consisting of between 100 to 400 nm wavelengths, between 100 and 300 nm wavelengths, between 100 and 200 nm wavelengths, between 200 and 400 nm wavelengths, between 200 and 300 nm wavelengths, and between 300 and 400 nm wavelengths may also be utilized.

At least one, and preferably each, of the at least one light source may be a light emitting diode. The light emitting diode(s) may be selected from the group consisting of at least one UVC light emitting diode, at least one UVA light emitting diode, and combinations thereof. In certain embodiments, the light source may further comprise a circuit board, wherein at least one of the light emitting diode(s) may be electrically connected to the circuit board. Preferably, each of the light emitting diode(s) is electrically connected to the circuit board.

The at least one light source mounted within the housing may be activated by a switching mechanism to which the at least one light source is electrically connected. A switching mechanism may be defined as an electrical device which sends an electrical signal to the at least one light source upon the initiation of an event. The event may be a user defined event, such as pushing a button or activating a switch to close a circuit which turns the at least one light source on (i.e.—activates the light source). Alternatively, the event may be a programmed event as described herein.

In some embodiments, the switching mechanism may be contained within the point-of-sale terminal. Some such switching mechanism may activate the at least one light source upon a programmed event. For example, the switching mechanism may be configured to send an electrical signal turning on the at least one light source upon completion of a transaction conducted on the point-of-sale terminal.

Certain embodiments of the switching mechanism may also comprise a timer. The timer—when used—may be configured to turn off the at least one light source after a first period of time. The first period of time may be in the range selected from the group consisting of between 10 seconds and 30 minutes after the at least one light source is turned on, between 10 second and 15 minutes after the at least one light source is turned on, between 10 seconds and 5 minutes after the at least one light source is turned on, between 10 seconds and 1 minutes after the at least one light source is turned on, between 1 minute and 30 minutes after the at least one light source is turned on, between 1 minute and 15 minutes after the at least one light source is turned on, between 1 minute and 5 minutes after the at least one light source is turned on, between 5 minutes and 30 minutes after the at least one light source is turned on, between 5 minutes and 15 minutes after the at least one light source is turned on, and between 15 minutes and 30 minutes after the at least one light source is turned on.

In some embodiments, the switching mechanism may comprise a passive infrared sensor. The passive infrared sensor may detect the presence of a person in the vicinity of the point-of-sale terminal by measuring infrared radiation signatures in and around the vicinity of the point-of-sale terminal. When the passive infrared sensor detects radiation levels indicative of the presence of a person in a predetermined location at or around the point-of-sale terminal, the passive infrared sensor may be configured to send an electrical signal turning off the at least one light source.

Like the light source mounted within the housing, the at least one secondary light source mounted within the cavity of the stylus holder may be any type of light source capable of emitting light in the ultraviolet spectrum. Preferably, the secondary light source is a UVGI light source designed to create a wavelength of light having disinfecting characteristics as described herein.

Wavelengths between about 200 nm and 300 nm are most preferred for the at least one secondary light source. However, light sources emitting UV light in a range selected from the group consisting of between 100 to 400 nm wavelengths, between 100 and 300 nm wavelengths, between 100 and 200 nm wavelengths, between 200 and 400 nm wavelengths, between 200 and 300 nm wavelengths, and between 300 and 400 nm wavelengths may also be utilized.

At least one, and preferably each, of the at least one secondary light source may be a light emitting diode. The light emitting diode(s) of the at least one secondary light source may be selected from the group consisting of at least one UVC light emitting diode, at least one UVA light emitting diode, and combinations thereof. In certain embodiments, the secondary light source may further comprise a circuit board, wherein at least one of the light emitting diode(s) of the secondary light source may be electrically connected to the circuit board. Preferably, each of the light emitting diode(s) is electrically connected to the circuit board.

Like the light source mounted within the housing, the at least one secondary light source mounted within the cavity may be activated by a secondary switching mechanism to which the at least one secondary light source is electrically connected. A switching mechanism may be defined as an electrical device which sends an electrical signal to the at least one secondary light source upon the initiation of an event. The event may be a user defined event, such as pushing a button or activating a switch to close a circuit which turns the at least one secondary light source on (i.e.—activates the secondary light source). Alternatively, the event may be a programmed event as described herein.

In some embodiments, the secondary switching mechanism may be contained within the point-of-sale terminal. Some such switching mechanism may activate the at least one secondary light source upon a programmed event. For example, the secondary switching mechanism may be configured to send an electrical signal turning on the at least one secondary light source upon completion of a transaction conducted on the point-of-sale terminal.

In other embodiments, the secondary switching mechanism may be in the form of a sensor which determines when the stylus is contained within the cavity. When the sensor detects that the stylus is contained within the cavity, the sensor closes a circuit which turns the at least one secondary light source on (i.e.—activates the secondary light source). When the sensor detects that the stylus has been removed from the cavity (i.e.—the stylus is not contained within the cavity), the sensor opens the circuit which turns the at least one secondary light source off (i.e.—deactivates the secondary light source).

In still other embodiments, the secondary switching mechanism may be in the form of a pressure switch located within the cavity. Preferably the pressure switch will be located in the bottom wall of the cavity, but may also be located at any point along the internal wall of the cavity. When the stylus is deposited into the cavity, the stylus contacts the pressure switch applying a pressure to the pressure switch which closes a circuit which turns the at least one secondary light source on (i.e.—activates the secondary light source). When the stylus is removed from the cavity, the pressure applied to the pressure switch by the stylus is released, opening the circuit and turning off the secondary light source (i.e.—deactivates the secondary light source). One example of a pressure switch is commonly known as a pin switch.

Certain embodiments of the secondary switching mechanism may also comprise a timer. The timer—when used—may be configured to turn off the at least one secondary light source after a first period of time. The first period of time may be in the range selected from the group consisting of between 10 seconds and 30 minutes after the at least one secondary light source is turned on, between 10 second and 15 minutes after the at least one secondary light source is turned on, between 10 seconds and 5 minutes after the at least one secondary light source is turned on, between 10 seconds and 1 minutes after the at least one secondary light source is turned on, between 1 minute and 30 minutes after the at least one secondary light source is turned on, between 1 minute and 15 minutes after the at least one secondary light source is turned on, between 1 minute and 5 minutes after the at least one secondary light source is turned on, between 5 minutes and 30 minutes after the at least one secondary light source is turned on, between 5 minutes and 15 minutes after the at least one secondary light source is turned on, and between 15 minutes and 30 minutes after the at least one secondary light source is turned on.

In some embodiments, the secondary switching mechanism may comprise a passive infrared sensor. The passive infrared sensor may detect the presence of a person in the vicinity of the point-of-sale terminal by measuring infrared radiation signatures in and around the vicinity of the point-of-sale terminal. When the passive infrared sensor detects radiation levels indicative of the presence of a person in a predetermined location at or around the point-of-sale terminal, the passive infrared sensor may be configured to send an electrical signal turning off the at least one secondary light source.

The system disclosed herein allows for disinfection of point-of-sale terminals without the need for user inputs, such as spraying a disinfectant onto the point-of-sale terminal and wiping. Additionally, the system disclosed herein ensures a more routine disinfecting of point-of-sale terminals by turning on the light source(s) at certain triggering events such as the completion of a transaction on the point-of-sale terminal; while at the same time reducing or preventing risk of ultraviolet radiation exposure to people in the vicinity of the disinfecting system by turning the light source off at certain triggering events such as the detection of a person in the vicinity of the point-of-sale terminal by a passive infrared sensor.

What is claimed is:

1. A system for disinfecting a point-of-sale terminal (10) said point-of-sale terminal comprising at least one planar display screen (12) and optionally at least one keypad, said system comprising:
a housing (100) comprising a first end wall (110), a second end wall (120), a first longitudinal wall (130) spanning a distance between a first end wall first edge (111) and a second end wall first edge (211), a second longitudinal wall (140) spanning a distance between a first end wall second edge (112) and a second end wall second edge (212), and an opening (150) extending from a second longitudinal wall first edge (141), said housing configured to connect to a first exterior surface (16) of the point-of-sale terminal; and
at least one light source (200); and
wherein the at least one light source is mounted within a hollow interior of the housing;
wherein the at least one light source is configured within the hollow interior of the housing to project light at a first angle relative to a first plane of the at least one planar display screen and/or the at least one keypad when the housing is connected to the first exterior surface of the point-of-sale terminal, wherein said first angle is in a range of between 0° and 90°;
wherein the at least one light source is electrically connected to a switching mechanism; and
wherein the at least one light source is capable of emitting light in the ultraviolet spectrum of between 100 nm and 400 nm upon receiving an electrical signal from the switching mechanism.

2. The system of claim 1, wherein the at least one light source comprises at least one light-emitting diode.

3. The system of claim 1, wherein the at least one light source is capable of emitting light in the ultraviolet spectrum of between 200 nm and 300 nm upon receiving the electrical signal from the switching mechanism.

4. The system of claim 1, further comprising a stylus holder (300) configured to connect to a second exterior surface (18) of the point-of-sale terminal, said stylus holder comprising:
a first end (310);
a second end (320) opposite the first end;
a sidewall (330) spanning the distance between the first end and the second end;
a first end access point (315);
a cavity (340) originating from the first end access point, said cavity configured to receive a stylus (400); and
at least one secondary light source (210); and
wherein the at least one secondary light source is mounted within the cavity;
wherein the at least one secondary light source is electrically connected to the switching mechanism; and
wherein the at least one secondary light source is capable of emitting light in the ultraviolet spectrum of between 100 nm and 400 nm upon receiving an electrical signal from the switching mechanism.

5. The system of claim 4, further comprising a secondary switching mechanism, the at least one secondary light source is electrically connected to the secondary switching mechanism, and the at least one secondary light source is capable of emitting light in the ultraviolet spectrum of between 100 nm and 400 nm upon receiving an electrical signal from the secondary switching mechanism.

6. The system of claim 5, wherein the secondary switching mechanism is contained within the point-of-sale terminal, and is configured to send an electrical signal turning on the at least one secondary light source upon completion of a transaction conducted on the point-of-sale terminal.

7. The system of claim 5, wherein the secondary switching mechanism comprises a sensor configured to send an electrical signal turning on the at least one secondary light source when the sensor detect the presence of the stylus within the cavity.

8. The system of claim 5, wherein the secondary switching mechanism comprises a pressure switch located within the cavity and configured to send an electrical signal turning on the at least one secondary light source when a pressure is applied to the pressure switch by the stylus.

9. The system of claim 5, wherein the secondary switching mechanism comprises a secondary passive infrared sensor configured to send an electrical signal turning off the at least one secondary light source when the secondary passive infrared sensor detects the presence of a person in the vicinity of the point-of-sale terminal.

10. The system of claim 5, wherein the secondary switching mechanism comprises a timer configured to turn off the at least one secondary light source after a first period of time in the range of between 10 seconds after the at least one secondary light source is turned on and 30 minutes after the at least one secondary light source is turned on.

11. The system of claim 4, wherein the at least one secondary light source is capable of emitting light in the ultraviolet spectrum of between 200 nm and 300 nm upon receiving the electrical signal from the switching mechanism.

12. The system of claim 4, wherein the point-of-sale terminal further comprises a stylus clip, and the stylus holder is configured to be held within the stylus clip.

13. The system of claim 4, wherein the sidewall comprises an adhesive exterior surface.

14. The system of claim 4, wherein the stylus holder is molded to the second exterior surface.

15. The system of claim 1, wherein the switching mechanism is contained within the point-of-sale terminal, and is configured to send an electrical signal turning on the at least one light source upon completion of a transaction conducted on the point-of-sale terminal.

16. The system of claim 1, wherein the switching mechanism comprises a passive infrared sensor configured to send an electrical signal turning off the at least one light source when the passive infrared sensor detects the presence of a person in the vicinity of the point-of-sale terminal.

17. The system of claim 15, wherein the switching mechanism comprises a timer configured to turn off the at least one light source after a first period of time in the range of between 10 seconds after the at least one light source is turned on and 30 minutes after the at least one light source is turned on.

18. The system of claim 1, wherein the housing comprises a bottom wall (160) comprising an adhesive exterior surface.

19. The system of claim 1, wherein the housing comprises an extension wall comprising an adhesive surface.

20. The system of claim 1, wherein the housing is molded to the first exterior surface.
